SEQUENCE LISTING (20 sequences)

SEQ ID NO: 1 - protein, 118 aa, Escherichia coli
MKKRGAFLGLLLVSACASVFAANNETSKSVTFPKCEDLDAAGIAASVKRDYQQNRVARWADDQKIVGQADPVAWVSLQDIQGKDDKWSVPLTVRGKSADIHYQVSVDCKAGMAEYQRR

SEQ ID NO: 2 - protein, 97 aa, Escherichia coli
ANNETSKSVTFPKCEDLDAAGIAASVKRDYQQNRVARWADDQKIVGQADPVAWVSLQDIQGKDDKWSVPLTVRGKSADIHYQVSVDCKAGMAEYQRR

SEQ ID NO: 3 - DNA, 357 bp, Escherichia coli
atgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgttaa

SEQ ID NO: 4 - DNA, 29 bp, Artificial
  PCR Primer
gagaattcggagaaaaacatgaaaaaaag

SEQ ID NO: 5 - DNA, 25 bp, Artificial
  PCR primer
atatctcgagacgccgctgatattc

SEQ ID NO: 6 - DNA, 43 bp, Artificial
  PCR primer
tagaattcaggagaaaaacatggtcgactcggtcaaaaacggg

SEQ ID NO: 7 - DNA, 25 bp, Artificial
  PCR primer
atatctcgagatgaggcgcatttcc

SEQ ID NO: 8 - DNA, 25 bp, Artificial
  PCR primer
atatctcgagagcgaaaactgatgc

SEQ ID NO: 9 - DNA, 27 bp, Artificial
  PCR primer
gatcatgtcgacgctccgacctccagc

SEQ ID NO: 10 - DNA, 25 bp, Artificial
  PCR primer
atatctcgagggtcagggtggagat

SEQ ID NO: 11 - protein, 133 aa, Homo sapiens [sequence omitted]

SEQ ID NO: 12 - DNA, 29 bp, Artificial
  PCR primer
gcatatctcgagcggacaccagaaatgcc

SEQ ID NO: 13 - DNA, 26 bp, Artificial
  PCR primer
cgatagtcgactttcagccccagagc

SEQ ID NO: 14 - protein, 21 aa, Escherichia coli
MKKRGAFLGLLLVSACASVFA

SEQ ID NO: 15 - protein, 21 aa, Shigella flexneri
MKKRGAFLGRLLVSACASVFA

SEQ ID NO: 16 - protein, 21 aa, Salmonella typhi
MNKRGALLSLLLLSASVSAFA

SEQ ID NO: 17 - protein, 23 aa, Yersinia pestis
MKKTGLALVLATILLGMMGSVHA

SEQ ID NO: 18 - protein, 27 aa, Pseudomonas luminescens
MKTCHIINRVGLSGVALLLTVSFTVSA

SEQ ID NO: 19 - DNA, 4347 bp, Artificial
  YebF expression vector
tgttgacaattaatcatcggctcgtataatgtgtggaattgtgagcggataacaatttcacacaggaaacagaattaggagatagaattcatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctcgagcaccaccaccaccaccactgagatctccgatcctctagagtcgatcgacctgcagccaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagc

SEQ ID NO: 20 - protein, 20 aa, Escherichia coli
MKKRGAFLGLLLVSACASVF

(19) United States
(12) Patent Application Publication
(10) Pub. No.: US 2006/0246539 A1
(43) Pub. Date: Nov. 2, 2006

(54) Protein production method utilizing yebF

(75) Inventors: Joel Weiner, Edmonton (CA); Guijin Zhang, Edmonton (CA)

Correspondence Address: EDWARD YOO C/O BENNETT JONES, 1000 ATCO CENTRE, 10035 - 105 STREET, EDMONTON, ALBERTA, AB, T5J3T2, CA

(73) Assignee: Governors of the University of Alberta

(21) Appl. No.: 11/203,168

(22) Filed: Aug. 15, 2005

Related U.S. Application Data
(60) Provisional application No. 60/522,125, filed on Aug. 18, 2004.

(51) Int. Cl. C07H 21/04; C12P 21/06; C12N 15/74; C12N 1/21; C07K 14/245

(52) U.S. Cl. 435/69.1; 435/252.33; 435/488; 530/350; 536/23.7

(57) ABSTRACT

A method of producing a protein or polypeptide, in which an expression vector that encodes a fusion protein comprising the protein or polypeptide peptide located carboxy-terminally to YebF, or a biologically active variant or portion thereof, is expressed in a suitable bacterial cell, to make the fusion protein. The method may further comprise the step of purifying the secreted fusion protein from the growth medium. The fusion protein may further comprise a peptide tag, or a protein cleavage site to separate the protein or polypeptide from the YebF portion, or the tag. Expression of a fusion protein into the culture medium provides a starting material for purification, in which the secreted fusion protein is relatively pure.

### FIELD

The present invention relates to the field of secretion of proteins by host cells, into a growth medium.

### BACKGROUND

Protein secretion from bacteria plays an important role in many aspects of the bacterial life cycle, including the formation of pili and flagella, the secretion of extracellular enzymes to digest polymers for nutritional purposes and the secretion of toxins to kill host cells in infections of humans, animals and plants. It is generally accepted that non-pathogenic laboratory strains of E. coli, particularly K12 strains, do not naturally secrete proteins into the extracellular medium under routine growth conditions^{6,8}, although gene sequence analysis implies the possible existence of a protein secretion pathway⁶. Pugsley has postulated that this system has been lost as a result of prolonged laboratory passage and storage over many decades⁶.

Bacillus subtilis has a well characterized protein excretion system for extracellular enzymes such as α-amylase and subtilin and this organism has been used as an alternative for the extracellular production of recombinant proteins⁷. Compared to E. coli, however, B. subtilis has several disadvantages including plasmid instability, lack of suitable expression control systems and degradation of the recombinant proteins in the culture medium by the high level of proteolytic enzymes that are also secreted from these cells⁷. In addition, compared with E. coli, a relatively long culture period is required for B. subtilis.

Other investigators have examined the secretion of proteins in E. coli. He et al.¹⁰ introduced a cluster of out genes (˜12 kb) carried on a cosmid to E. coli cells whose chromosome had Erwinia pectate lyase gene integrated. The out genes encode the secretion apparatus of Erwinia chrysnthemi, an enterobacterial plant pathogen. With the help of out gene expression, E. coli could secrete the enzyme to the medium. Pugsley's group⁸ has shown that E. coli cells can secrete Klebsiella oxytoca pullulanase into the medium by co-expressing 14 Klebsiella secretion machinery genes cloned on a second plasmid. Pugsley's group¹¹ has also shown that the secretion of an endogenous E. coli chitinase into the medium can occur by co-expressing a cluster of E. coli gsp genes carried on a second plasmid in PAP5066, an hns-inactivated E. coli K12 strain. The gsp genes encode the putative type II secretion machinery proteins of E. coli and are silenced by H-NS protein, a global regulator and nucleoid-structuring protein¹¹. Overall, these excretion studies required the artificial induction of secretion machinery proteins.

In E. coli K12, there are 2 well-identified protein transport pathways across the cytoplasmic membrane—the sec-dependent pathway or the general secretion pathway (GSP) and the sec-independent Mtt/Tat pathway¹². The GSP of gram-negative bacteria, has at least six different terminal branches depending on the secretion pathway in the outer membrane¹³. Genomic analysis of E. coli has identified genes that are homologous to genes encoding secretion proteins in other bacteria. These include gsp and yacC (gspS) homologous to Klebsiella pulS¹¹, found at 74.5 and 2.95 minutes on the E. coli chromosome, respectively. Although these genes appear to encode functional proteins¹⁴, their transcription is turned off under standard growth condition 6 gsp genes, for example, are silenced by H-NS as mentioned above

E. coli is the most widely used bacterium for protein expression both in research and in industry. As it is desirable to have a secretion system in common laboratory strains, investigators have explored different ways of producing recombinant proteins in the medium⁵. The accumulation of recombinant proteins in the culture medium has the potential to offer a number of benefits including, increased protein production and purity, reduction in cellular toxicity of over-expressed proteins, avoidance of the formation of inclusion bodies and protein degradation by cytoplasmic proteolytic enzymes. It has also been found that proper recombinant protein folding is enhanced by the more favorable redox potential in the medium⁵.

Based on sequence motif algorithms, the E. coli yebF gene is predicted to encode a small lipoprotein (Blattner B1847 or Swiss-Prot P33219) of unknown function attached to the membrane. The yebF gene is part of the yebGFE operon that is negatively regulated by LexA^{1,9}. yebF gene expression has been observed under several stress conditions including UV irradiation² and DNA damage induced by mitomycin C^{3,9}. However, little else has been reported.

### SUMMARY

YebF is a small (10.8 kD) soluble endogenous protein that is naturally secreted into the culture medium by E. coli cells. Commonly used laboratory strains HB101 (a hybrid of E. coli K12 and B strains), BL21(DE3) (a B strain) and MG1655 (a K12 strain) secrete YebF into the medium under standard laboratory conditions.

YebF can be used to transport proteins into the growth medium. Fusion proteins comprising YebF and human interleukin-2 (hIL2), the short form of B. subtilis X23 α-amylase gene⁸ lacking the signal sequence and leaderless E. coli alkaline phosphatase, have been expressed and secreted in E. coli. Therefore, YebF can be used to direct the secretion of proteins, polypeptides and peptides into the medium. Further, the secreted fusion proteins are active.

hIL2 is a 15 kD hydrophobic protein¹⁵, the short form of α-amylase is a 48 kD hydrophilic protein, and leaderless E. coli alkaline phosphatase is a hydrophilic protein. Therefore, YebF can direct the secretion of both hydrophobic and hydrophilic proteins to the medium. Further, YebF can carry proteins of varying size to the medium. However, it was noted that the secreted YebF-hIL2 fusion was sensitive to proteolytic degradation in the medium.

When the fusion protein is further engineered to comprise a peptide tag, such as a His6 tag, the secreted fusion protein can be readily purified from the medium, for example by affinity chromatography, or it can be readily identified, for example with a fluorescent antibody. Expression of a fusion protein into the culture medium provides a starting material for purification wherein the secreted fusion protein is relatively pure, as compared to proteins expressed in the cytoplasm. Therefore subsequent purification steps may be simpler and less expensive. Further, production of secreted fusion proteins in the medium could reduce the contamination of these proteins with lipopolysaccharides in the purified product¹⁶.

Therefore, in one aspect this invention is a method of producing a protein, polypeptide or peptide of interest that comprises:
(a) providing an expression vector that encodes a fusion protein comprising the protein, polypeptide or peptide of interest located carboxy-terminally to YebF, or a biologically active variant or portion thereof, and
(b) expressing the fusion protein in a suitable bacterial cell to thereby generate a secreted fusion protein.

The bacterial cell can be Escherichia coli. The method may further comprise the step of purifying a secreted fusion protein from the medium in which the bacterial cell is growing. The fusion protein may further comprise at least one tag or at least one protein cleavage site.

In another aspect the invention is a method of secreting a protein, polypeptide or peptide of interest from a bacterial cell into the growth medium comprising:
(a) making an expression vector that comprises a recombinant DNA molecule that further comprises a nucleotide sequence that encodes the protein, polypeptide or peptide of interest joined in frame to a nucleotide sequence that encodes YebF, or a biologically active variant or portion thereof,
(b) transfecting the expression vector into the bacterial cell, and
(c) expressing the protein encoded by the recombinant DNA molecule in the bacterial cell under conditions that direct the secretion of the protein into the growth medium.

The bacterial cell may be Escherichia coli. The recombinant DNA molecule may further comprise at least one nucleotide sequence that encodes a tag or a protein cleavage site.

In another aspect, the invention is the use of YebF, or a biologically active variant or portion thereof to direct the secretion of a protein, polypeptide or peptide of interest into bacterial growth medium.

The YebF, or a biologically active variant or portion thereof may be part of a fusion protein that further comprises the protein, polypeptide or peptide of interest. The YebF, or a biologically active variant or portion thereof may be fused amino-terminally to the protein, polypeptide or peptide of interest.

In yet another aspect, the invention is an expression vector that encodes YebF, or a biologically active variant or portion thereof operatively linked to control nucleotide sequences that direct the transcription and the synthesis of the YebF or a biologically active variant or portion thereof.

In yet another aspect, the invention is an expression vector that encodes a fusion protein comprising YebF, or a biologically active variant or portion thereof and a protein, polypeptide or peptide of interest. The nucleotide sequence that encodes the protein, polypeptide or peptide of interest may be located downstream of the nucleotide sequence that encodes YebF, or a biologically active variant or portion thereof. The expression vector may be for use in Escherichia coli. The expression vector may further encode a fusion protein that further comprises at least one tag.

### BRIEF DESCRIPTION OF THE DRAWINGS

FIG. 1. Alignment of the leader sequences of E. coli YebF protein and homologues from other bacteria.

FIG. 2. Western blot demonstrating the subcellular localization of E. coli YebF and mature YebF. Symbols: S, spheroplasts; P, periplasm; H, hypertonic solution; M, medium (Panel A). Western blot demonstrating secretion of mature YebF protein from E. coli cells over time. Lanes 1-7 show the cells (0-180 min) and lanes 8-14 show the medium (0-180 min) at 30 min time intervals. Lane F is purified YebF-His₆ (Panel B)

FIG. 3. Protein profiles in the medium from HB101 cells harbouring pMS119EH (Ctr lanes) or pYebFH₆/MS (YebF lanes). Samples were collected at the times indicated after induction with 0.1 mM IPTG. Proteins were separated on a 10% SDS-Tricine gel.

FIG. 4. Maldi-TOF linear mode mass spectrometry of purified E. coli YebF protein with a 6-histidine tag.

FIG. 5. Western blot demonstrating localization of YebF and mature YebF and CRP in cells (C) or medium (M), over time. YebF and mature YebF were detected with anti-His antibody (Panel A), CRP was detected with anti-CRP antibody (Panel B). Lane F in panel A is the purified YebF-His₆.

FIG. 6. Panel A: α-amylase activity of the various fluids and subcellular fractions of HB101 cells harboring: (a) a short form α-amylase gene fused to yebF gene (pYebF-AmyH₆), (b) a short form α-amylase gene fused to the sec-leader sequence of yebF (pLS-AmyH₆), and (c) a short form α-amylase gene (pAmyH₆). Data is the average of three replicates with the standard deviation shown.

Panel B: Protein profiles in the medium. HB101 cells harbouring pAmyH₆ (A lanes), pLS-AmyH₆ (B lanes) and pYebF-AmyH₆ (C lanes) were grown in TB. Protein samples from the medium harvested at the indicated time after induction were prepared as in Methods and Materials. Proteins were separated on 10% SDS-PAGE.

FIG. 7. Distribution of YebF-α-amylase and alkaline phosphatase, a periplasmic enzyme. Activities shown are the average of two determinations (Panel A). MIC (Minimum Inhibitory Concentrations) test of HB101 cells harboring the plasmids for the antibiotics indicated in the Table. (Panel B) pBR322-tet and pYebFH₆/MS-tet are tetracycline resistant. The remainder are amp-resistant.

FIG. 8. Western blotting demonstrating secretion of mature YebF from E. coli HB101/pYebFH₆/MS (HB), BL21(DE3)/pYebFH₆/T7 (BL) and MG1655/pYebFH₆/MS (MG) cells. Lane F, purified YebF-His₆.

FIG. 9. Western blotting demonstrating the secretion of mature YebF-hIL2 fusion protein from HB101 harboring pYebF-hIL2H₆/MS. Panel A: shows the localization of YebF-hIL2 by immunoblotting using an anti-6xhistidine tag antibody. Lane M1, medium+10 mM DTT; C1, cells+10 mM DTT; M2, medium−DTT; C2, cells−DTT.

Panel B shows the activities of the secreted mature YebF-hIL2 fusion protein in the diluted medium. The bar labeled “600 units/ml” corresponds to standard hIL-2 with the well containing 50 μl of 600 units of hIL-2/ml in RHFM medium (see Materials and Methods) and 50 μl of CTLL-2 cells. The bar labeled “negative” is a negative control in which RHFM without hIL-2 was used. The bar labeled “medium” corresponds to ten times diluted medium from HB101/pMS119EH. The E. coli culture medium harvested 3 hours after induction was filtered through a 0.22 μm Millipore filter and then diluted with RHFM at the ratios indicated. Each bar was averaged from triplicate wells.

FIG. 10. Secretion of mature YebF-α-amylase fusion protein over time from HB101 harboring pYebF-AmyH₆. Samples were taken at 2 hour intervals following 0.1 mM IPTG induction and assayed for α-amylase activity. Growth was monitored with a Klett spectrophotometer equipped with a red filter. α-amylase activity in the cells and the medium was expressed in units per ml of the original culture.

FIG. 11. The sequence of pYebFH6/MS. The ribosome binding site is in bold, and the YebF coding region with a 6-histidine tag is underlined.

FIG. 12. The physical map of pYebFH6/MS.

### DETAILED DESCRIPTION

Disclosed herein is a method for secreting a protein, polypeptide or peptide of interest into bacterial growth medium. Also disclosed herein is a method of producing a protein, polypeptide or peptide of interest. The method utilizes YebF, or a biologically active variant or portion thereof, to direct or cause the protein, polypeptide or peptide of interest, to be secreted as a fusion protein into the bacterial growth medium. The fusion protein comprises YebF, or a biologically active variant or portion thereof fused to a protein, polypeptide or peptide of interest. The fusion protein may optionally be tagged and may optionally comprise an amino acid sequence that permits cleavage of the YebF encoded portion and/or the tag, from the protein, polypeptide or peptide of interest.

To facilitate understanding of the invention, a number of terms are defined below.

The term “nucleotide” refers to a ribonucleotide or a deoxyribonucleotide. “Nucleic acid” refers to a polymer of nucleotides and may be single- or double-stranded. “Polynucleotide” refers to a nucleic acid that is twelve (12) or more nucleotides in length.

The term “nucleotide sequence of interest” refers to any nucleotide sequence that encodes a “protein, polypeptide or peptide sequence of interest”, the production of which may be deemed desirable for any reason, by one of ordinary skill in the art. Such nucleotide sequences include, but are not limited to, coding sequences of structural genes (e.g., reporter genes, selection marker genes, oncogenes, drug resistance genes, growth factor genes, etc.), regulatory genes (e.g., genes encoding activator protein 1 (AP1), activator protein 2 (AP2), Sp1, etc.), antibody genes, enzyme genes, etc., or portions thereof. The nucleotide sequence of interest may comprise the coding sequence of a gene from one of many different organisms (e.g., mammalian, insect, bacterial, and viral genes).

A nucleotide sequence “encodes” or “codes for” a protein if the nucleotide sequence can be translated to the amino acid sequence of the protein. The nucleotide sequence does not have to contain an actual translation start codon or termination codon.

A “protein, polypeptide or peptide sequence of interest” is encoded by the “nucleotide sequence of interest”. The protein, polypeptide or peptide may be a protein from any organism, including but not limited to, mammals, insects, micro-organisms such as bacteria and viruses. It may be any type of protein, including but not limited to, a structural protein, a regulatory protein, an antibody, an enzyme, an inhibitor, a transporter, a hormone, a hydrophilic or hydrophobic protein, a monomer or dimer, a therapeutically-relevant protein, an industrially-relevant protein, or portions thereof.

“Expression” as used herein refers to transcription or translation, or both, as context requires.

“Secretion” as used herein refers to the excretion of the fusion protein that is expressed in a bacterium, into the bacterial growth medium.

“YebF” is a reference to the protein having the following amino acid sequence (SEQ ID NO: 1)

MKKRGA FLGLLLVSAC ASVFAANNET SKSVTFPKCE DLDAAGIAAS
VKRDYQQNRV ARWADDQKIV GQADPVAWVS LQDIQGKDDK
WSVPLTVRGK
SADIHYQVSV DCKAGMAEYQ RR

“Mature YebF” is a reference to the protein having the following amino acid sequence (SEQ ID NO: 2)

ANNET SKSVTFPKCE DLDAAGIAAS VKRDYQQNRV ARWADDQKIV
GQADPVAWVS
LQDIQGKDDK WSVPLTVRGK SADIHYQVSV DCKAGMAEYQ RR

yebF is a reference to a nucleic acid or a nucleotide sequence having the following sequence (SEQ ID NO: 3):

atg aaa aaa aga ggg gcg
ttt tta ggg ctg ttg ttg gtt tct gcc tgc
gca tca gtt ttc gct gcc aat aat gaa acc
agc aag tcg gtc act ttc cca aag tgt gaa
gat ctg gat gct gcc gga att gcc gcg agc
gta aaa cgt gat tat caa caa aat cgc gtg
gcg cgt tgg gca gat gat caa aaa att gtc
ggt cag gcc gat ccc gtg gct tgg gtc agt
ttg cag gac att cag ggt aaa gat gat aaa
tgg tca gta ccg cta acc gtg cgt ggt aaa
agt gcc gat att cat tac cag gtc agc gtg
gac tgc aaa gcg gga atg gcg gaa tat cag
cgg cgt taa

The terms “modified”, “mutant” or “variant” are used interchangeably herein, and refer to: (a) a nucleotide sequence in which one or more nucleotides have been added or deleted, or substituted with different nucleotides or modified bases (e.g., inosine, methylcytosine) or to (b) a protein, peptide or polypeptide in which one or more amino acids have been added or deleted, or substituted with a different amino acid. A variant may be naturally occurring, or may be created experimentally by one of skill in the art. A variant of YebF or yebF may be a protein, peptide, polypeptide or polynucleotide that differs (i.e., an addition, deletion or substitution) in one or more amino acids or nucleotides from the sequence of YebF or yebF, respectively.

In this regard, it is well understood in the art that certain alterations inclusive of mutations, additions, deletions and substitutions can be made to a reference nucleic acid or protein, whereby the altered nucleic acid or protein retains a particular biological function or activity, or perhaps displays an altered but nevertheless useful activity. Some deletions, insertions and substitutions will not produce radical changes in the characteristics in the YebF protein or in the YebF nucleic acid. However, while it may be difficult to predict the exact effect of the substitution, deletion or insertion in advance of doing so, one skilled in the art will appreciate that the effect can be evaluated by routine screening assays. For example whether a variant of YebF has a secretory function can be determined by assaying for whether the YebF variant, or a fusion protein comprising the YebF variant, is secreted into the medium, by the methods disclosed in the Materials and Methods, and Examples disclosed herein. Modifications of protein properties such as redox or thermal stability, hydrophobicity, susceptibility to proteolytic degradation, or the tendency to aggregate with carriers or into multimers may be assayed by methods well known to one of skill in the art.

Variants may be created experimentally using random mutagenesis, oligonucleotide-mediated (or site-directed) mutagenesis, PCR mutagenesis and cassette mutagenesis. Oligonucleotide-mediated mutagenesis is well known in the art as, for example, described by Adelman³¹ using vectors that are either derived from bacteriophage M13, or that contain a single-stranded phage origin of replication as described by Viera et al.³² Production of single-stranded template is described, for example, in Sambrook.¹⁷ Alternatively, the single-stranded template may be generated by denaturing double-stranded plasmid (or other DNA) using standard techniques.

Alternatively, linker-scanning mutagenesis of DNA may be used to introduce clusters of point mutations throughout a sequence of interest that has been cloned into a plasmid vector. For example, reference may be made to Ausubel.³³ Region-specific mutagenesis and directed mutagenesis using PCR may also be employed to construct variants according to the invention. In this regard, reference may be made, for example, to Ausubel.³³ With regard to random mutagenesis, methods include incorporation of dNTP analogs³⁴ and PCR-based random mutagenesis such as described in Stemmer and Shafikhani.^{35,36}

The term “biologically active” when made in reference to a variant or portion of YebF, refers to a protein, polypeptide or peptide possessing at least the secretory function of YebF. A biologically active variant or portion of YebF has at least about 5%, preferably at least about 25%, more preferably at least about 50% and most preferably at least about 75% of the secretory activity of YebF. Whether the variant or portion of the YebF protein has secretory activity may be determined, for example, by the methods disclosed in the Materials and Methods, and Examples disclosed herein. A biologically active variant or portion of YebF may either be secreted into the growth medium, or may effect the secretion a fusion protein (of which it comprises part) into the growth medium, and both of these activities are considered to be a secretory activity.

The term “portion” when used in reference to a protein refers to fragments of that protein. The fragments may range in size from four amino acid residues to the entire amino acid sequence of the protein, minus one amino acid.

A “peptide” is polymer of four to 20 amino acids, a “polypeptide” is a polymer of 21 to 50 amino acids and a “protein” is a polymer of more than 50 amino acids.

A fusion protein is a recombinant protein comprising regions derived from at least two different proteins. The term “fusion protein” as used herein refers to a protein molecule in which a protein, polypeptide or peptide of interest is fused to: YebF, a biologically active variant of YebF, or a biologically active portion of YebF (herein a “YebF, or a biologically active variant or portion thereof”). “Fused”, in one context means that nucleic acid encoding YebF, or a biologically active variant or portion thereof, is joined in frame to the nucleic acid encoding the protein, polypeptide or peptide of interest, to provide for a single amino acid chain when transcription and translation occur. In another context, “fused” may also be a reference to the joining of a protein, polypeptide or peptide of interest to YebF, or a biologically active variant or portion thereof.

A “secreted fusion protein” is the part of the fusion protein that is secreted into the bacterial growth medium. As is apparent, a secreted fusion protein will likely lack the amino acids that comprise the leader sequence of YebF, specifically MKKRGA FLGLLLVSAC ASVF.

An “expression vector” refers to a recombinant DNA molecule containing the appropriate control nucleotide sequences (e.g., promoters, enhancers, repressors, operator sequences and ribosome binding sites) necessary for the expression of an operably linked nucleotide sequence in a particular host cell. By “operably linked/linking” or “in operable combination” is meant that the nucleotide sequence is positioned relative to the control nucleotide sequences to initiate, regulate or otherwise direct transcription and/or the synthesis of the desired protein molecule.

The expression vector may be self-replicating, such as a plasmid, and may therefore carry a replication site, or it may be a vector that integrates into a host chromosome either randomly or at a targeted site. The expression vector may contain a selection gene as a selectable marker for providing phenotypic selection in transformed cells. The expression vector may also contain sequences that are useful for the control of translation.

“Purified” or “to purify” refers to the removal of undesired components from a sample. For example, to purify the secreted fusion protein from the bacterial growth medium, may mean to remove other components of the medium (i.e., proteins and other organic molecules) thereby increasing the percentage of the secreted fusion protein.

The present invention utilizes YebF, or a biologically active variant or portion thereof, to direct the secretion of a protein, polypeptide or peptide of interest, into the bacterial growth medium. This may be accomplished by generating a fusion protein, which comprises YebF or a biologically active variant or portion thereof, and the protein, polypeptide or peptide of interest.

A recombinant DNA molecule that encodes the fusion protein can be made, for example, by ligating a nucleic acid that encodes the protein, polypeptide or peptide of interest (a nucleotide sequence of interest), in frame, to a nucleic acid that encodes the YebF protein, or a biologically active variant or portion thereof. In one embodiment, the nucleic acid that encodes the protein, polypeptide or peptide of interest may be ligated to the 3′-end (i.e., downstream) of the nucleic acid that encodes the YebF protein, or a biologically active variant or portion thereof. In another embodiment, the nucleic acid that encodes the protein, polypeptide or peptide of interest may not be ligated directly to the 3′-end of the YebF protein, or a biologically active variant or portion thereof. Rather, a spacer nucleotide sequence may separate the 3′-end of the nucleotide sequence encoding the YebF protein, or a biologically active variant or portion thereof, and the 5′-end of the nucleotide sequence that encodes the protein, polypeptide or peptide of interest. The spacer nucleotide sequence encodes one or more amino acids that may or may not be functional (i.e., a tag, or a cleavage site, or no function at all but to separate the two parts). Therefore, in the fusion protein produced by these embodiments, the protein, polypeptide or peptide of interest is located carboxy-terminally to the YebF protein, or a biologically active variant or portion thereof. Methods of generating this recombinant DNA molecule are known to those of skill in the art, examples of which are provided by the methods disclosed in the Materials and Methods, and Examples disclosed herein, or the references cited herein.

The nucleotide sequence that encodes the YebF protein, or a biologically active variant or portion thereof, or the nucleotide sequence that encodes the protein, polypeptide or peptide of interest may additionally comprise transcriptional and translational control regions, which are short arrays of DNA sequences that interact specifically with cellular proteins involved in transcription and translation. These regions include promoters, operator sequences and ribosome binding sites, and are known to those of skill in the art.

It may also be desirable to tag the fusion protein or the secreted fusion protein with at least one tag that may be useful for purifying the secreted fusion protein from the growth medium, for identifying the secreted fusion protein, or for some other purpose. Useful tags include epitope tags that are recognized by a specific antibody, thereby permitting the fusion protein to be purified by affinity chromatography, or to be identified, for example with a fluorescent antibody. Examples of a useful eptiopes are: glutathione-S-transferase, c-myc, poly-histidine, FLAG®, maltose binding protein, influenza A virus haemagglutinin, β-galactosidase and GALA, or portions thereof. Preferred may be a polyhistidine (poly-His tag). Cognate antibodies that recognize these epitope tags are known to those of skill in the art. For example, polyhistidine is recognized by an anti-His Mab available from Qiagen, FLAG® is recognized by Anti-FLAG M1, M2 and M5 Mabs, available from Sigma/Kodak. Therefore, contemplated herein may be the inclusion, in the recombinant DNA molecule, of at least one nucleotide sequence that encodes a tag amino acid sequence. The tag may be anywhere on the fusion protein, with the possible exception of at the amino-terminal end, or in the leader sequence, of the YebF protein, or a biologically active variant or portion thereof.

It may also be desirable to later cleave the secreted fusion protein, in order to separate the YebF portion (i.e., the mature YebF protein, or a biologically active variant or portion thereof) and/or the tag, from the protein, polypeptide or peptide of interest. For example, if the YebF portion and/or tag interferes with the biological activity of the protein, polypeptide or peptide of interest, it may be preferred to cleave either or both of them from the fusion protein. Therefore, contemplated herein may be the inclusion, in the recombinant DNA molecule, of at least one nucleotide sequence that encodes an amino acid sequence, which amino acid sequence facilitates the cleavage of the YebF portion and/or epitope tag and/or the protein, polypeptide or peptide, from the fusion protein. Examples of suitable amino acid sequences for cleavage by specific proteases are: (a) Factor Xa Protease—recognizes the amino acid sequence Ile-Glu-Gly-Arg and cleaves the peptide bond C-terminal of the arginine residue^{24,25}; (b) Tobacco Etch Virus (TEV) NIa protease—recognizes a seven amino acid consensus sequence, Glu-X-X-Tyr-X-Gln/Ser, where X can be various amino acyl residues, and cleaves between the conserved Gln and Ser residues²⁶; (c) PreScission™ Protease—cleaves between the Gln and Gly residues of the recognition sequence of LeuGluValLeuPheGln/GlyPro^{27,28}; (d) Thrombin—which is used to digest fusion proteins prepared from pGEX vectors (GST Gene fusion) containing the recognition sequence for thrombin²⁹, and (e) Enterokinase—which cleaves after lysine at its cleavage site Asp-Asp-Asp-Asp-Lys, or other basic residues, depending on the conformation of the protein substrate³⁰.

Therefore, a recombinant DNA molecule contemplated herein comprises a nucleotide sequence that encodes the YebF protein, or a biologically active variant or portion thereof and a nucleotide sequence that encodes the protein, polypeptide or peptide of interest, and optionally at least one nucleotide sequence that encodes a tag, and/or optionally at least one nucleotide sequence that encodes a protein cleavage site. These various nucleotide sequences are in frame with one another. Therefore, the fusion protein and secreted fusion protein contemplated herein comprise optionally, at least one tag and/or optionally at least one cleavage site.

The resultant recombinant DNA molecule may then be inserted into an expression vector. Useful vectors for practicing the invention disclosed herein are plasmids, for example pMS119EH, which comprises a lac promoter, and pT7-5, which comprises a T7 promoter. The expression vector selected will depend on which bacterial strain or species is to be used to express the fusion protein.

As is apparent to those of skill in the art, the above sequence of events need not be practiced in the above order, nor need the above methods be used, in order to generate the expression vector comprising the recombinant DNA molecule. For example, the nucleic acid that encodes the protein, polypeptide or peptide of interest may be inserted into the expression vector, followed by the insertion of the nucleic acid that encodes the YebF protein, or a biologically active variant or portion thereof, or vice versa (see Materials and Methods, herein). A nucleic acid that encodes the tag or the protein cleavage site, if used, may be inserted before or after either of the above components of the recombinant DNA molecule are inserted into the expression vector. The expression vector may already comprise a nucleic acid that encodes a tag sequence, obviating the need to add this component separately (see Materials and Methods, herein).

The expression vector comprising the recombinant DNA molecule is then transfected into the appropriate bacterial host, for expression of the fusion protein. Examples of how this can be accomplished are provided examples provided in the Materials and Methods, and Examples herein. As non-limiting examples, the expression vector may be transfected into HB101, BL21 or MG1655. For example, pMS119EH comprising an insert can be used in HB101 or MG1655, and pT7-5 comprising an insert can be used in BL21.

Preferred for use herein may be an expression vector that replicates extrachromosomally, as a greater number of copies of the fusion protein nucleic acid construct can be generated, which will generally result in higher levels of expression and hence secretion.

The present invention contemplates the secretion of many different proteins, polypeptides or peptides of interest. Contemplated herein are peptides as small as 4 amino acids. Also contemplated herein is any protein or polypeptide that can be secreted by the methods disclosed herein, regardless of the size. In the Examples provided, a protein of 48 kD was secreted, suggesting that large proteins can readily be secreted by the method disclosed herein.

Both hydrophilic and hydrophobic proteins may be secreted by the method disclosed herein. In the Examples provided both a hydrophobic protein (hIL2) and a hydrophilic protein (α-amylase or alkaline phosphatase) were secreted.

The bacterial cells are grown in growth medium, such as terrific broth, until such time as is desired to harvest the secreted fusion protein from the medium. The time required depends upon a number of factors relating to the bacterial expression system being used and to the fusion protein being produced. The rate of growth of a particular bacterial strain or species, the rate at which the secreted fusion protein accumulates in the medium, the stability of the secreted fusion protein in the medium, and the time at which bacterial lysis begins to occur (which will contaminate the growth medium) are non-limiting examples of the types of considerations that will affect when the secreted fusion protein is harvested from the culture medium.

Depending upon the level of purity required, which will again depend upon the application for which the secreted fusion protein will be used, the secreted fusion protein may be further purified, for example by affinity chromatography. If desired, the YebF portion and/or the tag may be cleaved from the secreted fusion protein, in order to generate the protein, polypeptide or peptide of interest, alone.

Disclosed herein is the fact that YebF can be used to direct the secretion of proteins, polypeptides and peptides, as fusion proteins, into the medium. The inventors have cloned three different nucleotide sequences of interest into a vector that comprises the nucleotide sequence for yebF. Accordingly, disclosed herein is an expression vector that comprises a nucleic acid encoding YebF operatively linked to control nucleic acid sequences needed to initiate, regulate or otherwise direct transcription and the synthesis of YebF. The expression vector may comprise restriction endonuclease sites, or other sites, downstream of the YebF coding region, useful for easy in frame insertion of a nucleic acid encoding a protein, polypeptide or peptide of interest. The expression vector may also comprise a tag nucleotide sequence, as shown in the Materials and Methods herein, or a nucleotide sequence encoding a protein cleavage site.

Also disclosed herein is the fact that the leader sequence of YebF may be used to direct the transport of a protein into the periplasm. Accordingly, disclosed herein is an expression vector that comprises a nucleic acid encoding the leader sequence of YebF operatively linked to control nucleic acid sequences needed to initiate, regulate or otherwise direct transcription and the synthesis of the YebF leader sequence. The expression vector may comprise restriction endonuclease sites, or other sites, downstream of the YebF leader coding region, useful for easy in frame insertion of a nucleic acid encoding a protein, polypeptide or peptide of interest. The expression vector may also comprise a tag nucleotide sequence, as shown in the Materials and Methods herein, or a nucleotide sequence encoding a protein cleavage site. Also disclosed herein is the use of the YebF leader sequence to direct transport of a protein, peptide or polypeptide to the periplasm. Also disclosed herein is a method for expressing a protein, polypeptide or peptide of interest in the periplasm, which method comprises providing an expression vector that encodes a protein comprising the protein, polypeptide or peptide of interest located carboxy-terminally to YebF leader sequence, and expressing the protein in a suitable bacterial cell.

While the invention has been described in conjunction with the disclosed embodiments, it will be understood that the invention is not intended to be limited to these embodiments. On the contrary, the invention is intended to cover alternatives, modifications and equivalents, which may be included within the spirit and scope of the invention as defined by the appended claims. Various modifications will remain readily apparent to those skilled in the art. Examples provided above and below are not intended to be limited to those examples alone, but are intended only to illustrate and describe the invention rather than limit the claims that follow.

### EXAMPLES

Materials and Methods

Bacterial strains and growth conditions: The E. coli strains used in this study were HB101 (supE44 hsd20(r_{B}⁻m_{B}⁻) recA13 ara-14 proA2 lacY1 gaIK2 rpsL20 xyl-5 mtl-1), BL21(DE3)(hsdS gal(λcIts857 ind1 Sam7 nin5 lacUV5-T7 gene 1), and MG1655(F-λ-ilvG-rfb-50 rph-1). Cells were grown at 30° C. in Terrific broth (TB). Antibiotics were added at the following concentrations: 100 μg/ml of ampicillin (Amp) and 80 μg/ml chloramphenicol (Cm). Expression of genes under lac promoter control was induced with 0.1 mM isopropyl-μ-D-thiogalactopyranoside (IPTG).

Molecular biology techniques and plasmid construction: DNA manipulation, sequencing and bacterial transformation were essentially as described¹⁷. Expression plasmids were constructed based on pMS119EH¹⁸ containing the lac promoter, or pT7-5 with the T7 promoter. PCR amplifications using the touch-down protocol and DNA purification thereafter were carried out as described¹⁹.

The yebF gene was amplified from HB101 chromosomal DNA using the following forward and reverse oligomers, respectively, with the indicated restriction sites underlined: yebF5, 5′-GAGAATTCGGAGAAAAACATGAAAAAAAG-3′ containing EcoR1 (SEQ ID NO: 4); and yebF3, 5′-ATATCTCGAGACGCCGCTGATATTC-3 (Xho1) (SEQ ID NO: 5). To tag the gene with 6 histidines at its 3′-end (yebFH₆), the amplified DNA, after digestion with EcoR1 and Xho1, was cloned into pCITE-2a(+) (Novagen, US) cut with the same enzymes. The EcoR1—Hind III fragment carrying yebFH6 was then subcloned into pMS119EH to generate pYebFH₆/MS, or into pT7-5 to make pYebFH₆/T7.

The mature truncated form of the α-amylase gene (GenBank accession no. AB015592) of Bacillus subtilis X-23⁴ was amplified from pAC92 kindly provided by Dr. Spartaco Astolfi-Filho, University of Brasilia, Brazil using the following oligos: amy5, 5′-TAGAATTCAGGAGAAAAACATGGTCGACTCGGTCA AAAACGGG-3′ (SEQ ID NO: 6) engineered with an EcoRI site, a ribosomal binding site (bold), the in-frame initiation codon ATG and SalI; amy3, 5′-ATATCTCGAGATGAGGCGCATT TCC-3 (XhoI) (SEQ ID NO: 7). The amplified DNA was cut with EcoRI and XhoI and replaced the yebF fragment (EcoRI-XhoI) in pYebFH6/MS to create pAmyH₆.

pYebF-AmyH₆ containing the yebF-amy fusion gene was created by replacing the EcoR1-Sal1 fragment in pAmyH₆ with the EcoR1-Xho1 fragment carrying the yebF gene from pYebFH₆/MS. Between the yebF nucleotide sequence and the amy nucleotide sequence, there is encoded an additional two amino acids—LE—which will link the two proteins.

To fuse the sec-leader sequence of the yebF gene to the mature α-amylase gene, the leader sequence was first amplified by PCR using pYebFH₆/MS as the template with Fls (5′-ATATCTCGAGAGCGAAAACTGATGC-3′) (SEQ ID NO: 8) containing an Xho1 restriction site and yebF5 (above) as the oligomers. After digestion with EcoR1 and Xho1, the amplified DNA fragment was inserted into pAmyH₆ to replace the EcoR1-Sal1 sequence, which generated pLS-AmyH₆. The sequence of this hybrid gene was verified by sequencing.

To construct pYebF-hIL2H₆, the hIL-2 gene was amplified by PCR from pBM806, a plasmid containing an hIL-2 gene provided by Biomira Inc. (Edmonton, Canada) using the following oligonucleotides: hil5 with Sal1, GATCATGTCGACGCTCCGACCTCCAGC (SEQ ID NO: 9) and hil3 with Xho1, ATATCTCGAGGGTCAGGGTGGAGAT (SEQ ID NO: 10). The Sal1-Xho1 fragment of the PCR product was inserted into Xho1 site in pYebFH6/MS to generate pYebF-hIL2H₆. Between the yebF nucleotide sequence and the hIL-2 nucleotide sequence, there is encoded an additional two amino acids—LE—which will link the two proteins, which is then followed by the six histidine residues as the tag.

The amino acid sequence of mature hIL-2 (i.e., lacking the 20 amino acid leader sequence) is:

(SEQ ID NO: 11)

APTSSSTKKT QLQLEHLLLD LQMILNGINN YKNPKLTRML
TFKFYMPKKA
TELKHLQCLE EELKPLEEVL NLAQSKNFHL RPRDLISNIN
VIVLELKGSE
TTFMCEYADE TATIVEFLNR WITFCQSIIS TLT

To make pYebF-phoAH₆/T7 plasmid, the phoA gene was PCR-amplified with the following oligos: phoA5 with XhoI, GCATATCTCGAGCGGACACCAGAAATGCC (SEQ ID NO: 12); and phoA3 with Sal1, CGATAGTCGACTTTCAGCCCCAGAGC (SEQ ID NO: 13). The XhoI-SalI fragment of the PCR product was inserted into XhoI site in pYebFH₆/T7 to generate pYebF-phoAH₆/T7.

Protein expression and treatment of samples: A single colony from LB plates or a small aliquot of cold cell suspension stored in 40% glycerol at −20° C. was inoculated into 1.5 ml of TB and grown overnight at 30° C. After harvesting by centrifugation the cells were washed once in 1.5 ml of fresh TB, and were suspended in 1.5 ml of fresh TB and inoculated at 1% by volume into fresh TB for expression studies. Protein expression was induced by addition of 0.1 mM IPTG or 0.5 mM IPTG at 0.6 OD₆₀₀. Cells were harvested at the indicated time points in the experiments. After harvesting by centrifugation at room temperature to avoid cold shock, cell pellets were washed once with a volume equal to the original culture volume of 100 mM MOPS (pH7.0). The resulting medium supernatant was filtered using a 0.22 μm Millipore filter to remove un-pelleted cells.

Subcellular fractionation: Periplasm was separated from cytoplasm by the osmotic shock method.²¹

α-amylase activity assay: This assay is based on the 3,5-dinitrosalicylic acid method as described by Ohdan et al.⁸ using starch as the substrate for the enzyme. The reaction was stopped by adding 200 μl of DNSA in 0.4N NaOH to 200 μl of the reaction mixture after 10 minutes incubation at 58° C. with shaking once per minute. The mixture was boiled for 5 minutes thereafter, diluted with 1 ml of water, and spun for 1 minute to pellet starch. Finally, readings were taken at 540 nm. The enzyme activity was expressed in moles of glucose released per minute that is equivalent to the amount of reducing sugar released by the enzyme from starch in the reaction. A pair of every sample to be assayed was set up. One is for the reaction, the other for background reading, to which 200 μl of DNSA was added before the reaction starts.

To measure the activity in the medium or in the osmotic shock fluid, the liquid was directly added to the reaction mixture. To assay the activity in cells or in the resulting spheroplasts from the osmotic shock, the cell suspension or the spheroplast suspension in 10 mM Tris-HCl (pH7.0) was mixed with equal volume of lysis buffer (pH 8.0) consisting of 70 μg/ml lysozyme, 6 mM EDTA, 2% Triton X-100 to break cells or spheroplasts. After lysis was complete on ice, 1 μl of 210 mM CaCl₂ was added to every 70 μl of the lysate to overcome the EDTA and allow for 0.1 mM Ca²⁺ in the reaction mixture as required for α-amylase activity.

Alkaline phosphatase activity assay: 1 ml of the reaction consists of 100 mM Tris-HCl (pH8.0), 10 mM MgCl₂, 2 mM ZnCl₂ and 0.04% pNPP. The reaction after incubation of indicated period was stopped by addition of 40 μl of 5M NaOH followed by 1 minute of centrifugation at 14,900 rpm before taking readings at 410 nm. To measure the background readings, samples were added to the mixtures after the reactions were stopped by NaOH. To calculate the enzyme activities, ε₄₁₀=17,800 M⁻¹ cm⁻¹ for p-nitrophenolate anion (pNP), the yellow product released from pNPP.

Purification of YebFH₆ and protein chemistry: Total protein in the culture medium of E. coli HB101/pYebFH6/MS grown overnight (15 hr) after induction with IPTG was precipitated by ammonium sulfate at 70% saturation at 4° C. in the presence of 0.2 mM PMSF. The pellet was dissolved in 20 mM Tris-HCl (pH7.5) containing 0.2 mM PMSF, 10 mM imidazol and 500 mM NaCl. Purification of YebFH₆ was achieved using a nickel affinity column (Amersham). This partially purified protein was further purified using a reversed-phase HPLC on a 300 Å C8 column (1 mm×15 cm) eluted with a gradient of acetonitrile from 0% to 2% in the presence of 0.05% TFA at the flow rate of 100 μl/min. A single major peak was observed at the 25th min. This peak fraction was collected and used for both MALDI-TOF linear mode mass spectrometry (Voyager 6064, Applied Biosystems) and N-terminal amino acid sequence determination using Routine 3.0 on a Hewlett Packard G1000A Protein Sequencer.

Protein electrophoresis and Western blotting: The protein samples from either the culture medium or the cells were prepared by precipitation with 5% trichloroacetic acid (TCA). After centrifugation in an Eppendorf tube at 14,900 rpm at room temperature for 5 minutes, the pellet was washed once by suspending it in 1 ml of water and centrifuging for 5 minutes to remove residual TCA. To almost completely extract TCA from the pellets, 1.5 ml of −20° C. cold acetone was added to each tube. The tube was then vortexed and kept at −20° C. for 20 minutes before centrifugation as above. The pellet was retained and subjected to one additional acetone wash. The final pellet was dissolved in 50 mM Tris-HCl, pH 8.0 before adding an equal volume of electrophoresis loading buffer. Proteins were separated on 8.5% SDS-Tricine gel²² and then transferred to a nitrocellulose membrane for immunoblotting. To detect the histidine tag, monoclonal mouse antibody against histidine tag (Qiagen) was used. CRP was detected using a rabbit polyclonal antiserum against CRP, kindly provided by Dr. Hiroji Aiba, Nagoya University, Japan. Immunoblots were developed by enhanced chemiluminescence using the ECL kit (Amersham).

IL-2 bioassay: The biological assay of YebF-hIL2 was based on the Lei et al. method²³ using the IL-2 dependent T lymphocyte cell line CTLL-2 with modifications. Prior to the assay, the YebF-hIL2 sample was prepared by passing the E. coli culture medium harvested 3 hours after induction with 0.1 mM IPTG from either HB101/pYebF-hIL2H₆ or pMS119EH (control) through a 0.22 μm Millipore filter. The cell culture medium (RHFM) for CTLL-2 uses RPMI 1640 supplemented with 10% (v/v) fetal calf serum, 0.1 mM β-mercaptoethanol, 25 mM HEPES pH7.5, 2 mM L-glutamine. 50 μl of fresh CTLL-2 cells (10⁶ cells/ml) in RHFM were placed in a well of a 96-well plate. 50 μl of standard hIL-2 (kindly provided by Dr. C. Bleackley, University of Alberta) or YebF-hIL2 samples diluted with RHFM was added. The cells were incubated at 37° C. with 5% CO₂ for 20 hours. Cell proliferation was measured using the MTT (3-(4,5-dimethylthiazol-2-yl)-2,5-diphenyl tetrazolium bromide) assay. 5 mg of MTT/ml was prepared in phosphate buffered saline and sterilized. 10 μl of MTT was added to each well followed by 4 hours incubation at 37° C. 150 g of isopropanol containing 0.04N HCl was added to each well and mixed thoroughly with a pipettor to dissolve the resulting crystals. The absorbance was read at 560 nm. Each value is the average of three assays.

Characterization of the E. coli YebF Leader

Examination of the DNA sequence of the yebGFE operon revealed a typical ribosome-binding site (GGAG, Blattner coordinates 1928421-1928424 in MG1655) upstream from the second ATG (1928412-1928414) in the region between yebG and yebF. Although the first ATG (Blattner coordinates 1928424-1928426) could initiate in-frame translation of YebF, and this is the way it is reported in databases such as NCBI (B1847) and Swiss-Prot (P33219), no ribosome-binding site could be found preceding this ATG. The yebF gene was cloned in the expression vector pMS 119EH under lac promoter control starting from nucleotide G (Blattner coordinate 1928424) without addition of a ribosome binding site and with the addition of a 6-histidine tag at the carboxyl terminus. The His-tagged YebF protein was positively expressed as shown by immunoblotting (see below).

The N-terminal amino acid sequence of E. coli YebF is as follows:
(−21)MKKRGAFLGLLLVSACASVFA(−1) (1)ANNE . . .

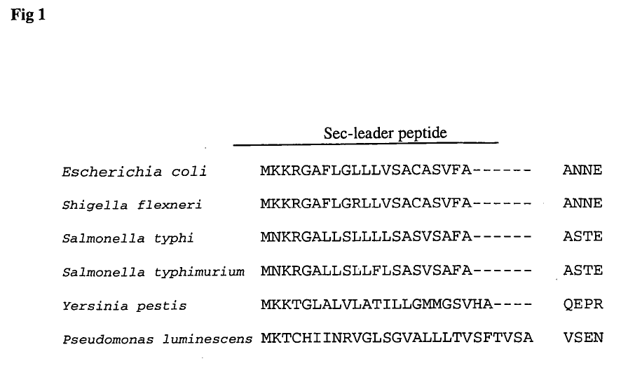

This sequence resembles the motif characteristic of lipoproteins⁴, including the fatty acid-acylation site (Cys-6). This motif is also conserved in YebF from Shigella flexneri (97% identity). However, this Cys is missing in other similar proteins such as STY2087 from Salmonella typhi (77% identity), YebF from Salmonella typhimurium (76% identity), YP01779 from Yersinia pestis (43% identity) and PLU2692 from Photorhabdus luminescens (40% identity). Therefore, YebF may not be a lipoprotein as suggested in several databases (B1847 at NCBI and P33219 at Swiss-Prot). The amino terminal sequence of the E. coli YebF protein also contains a typical 21 amino acid sec-leader peptide including 3 basic residues (−20 Lys-Lys-Arg-18) at the N-terminal end, followed by a 16 residue long hydrophobic core and a small residue (Ala-1) which could serve as the leader peptidase I cleavage site (FIG. 1). A similar leader sequence was found in all YebF related proteins (FIG. 1) suggesting that E. coli YebF may be secreted to the periplasm.

YebF Localization

To determine where YebF was localized, a 200 ml culture of HB101/pYebFH6/MS was grown in TB at 30° C. until the optical density at 600 nm reached 0.6. The culture was induced with 0.1 mM IPTG and harvested 4 hours after induction and washed once at room temperature with 200 ml of 100 mM MOPS (pH7.0). Cells were subjected to cold osmotic shock (Neu and Heppel, 1965) to separate periplasm from the cytoplasm. Protein samples from these resulting fractions and immunoblotting were prepared as described in Materials and Methods.

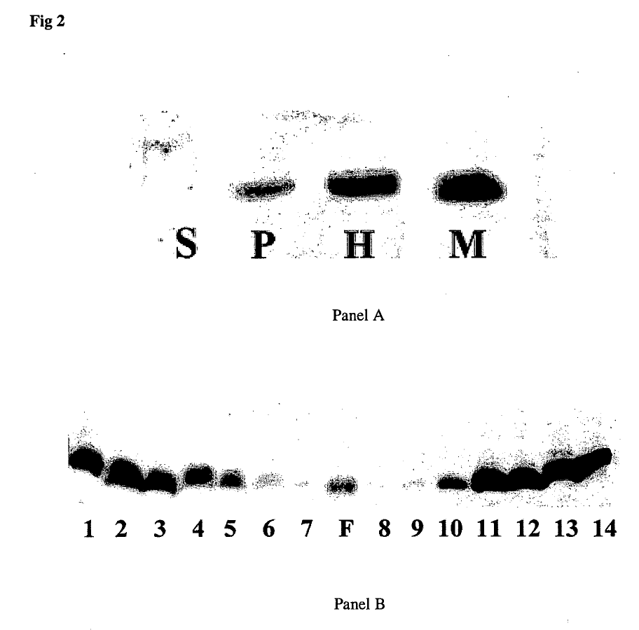

YebF was located in the periplasm and surprisingly also in the culture medium. No YebF was detected in the spheroplasts after cold osmotic shock (FIG. 2A).

To determine the effect of inhibition of protein synthesis on secretion, 10 ml HB101/pYebFH6/MS cells were grown as described for FIG. 2A. After harvesting, cells were washed once with 35 ml of fresh TB and suspended in 10 ml of fresh TB containing 80 μg chloramphenicol/ml. 1.5 ml of cells were taken at the time points indicated in FIG. 2B. Cells and medium were separated by centrifugation at room temperature to avoid cold shock. Immunoblotting was conducted as described in the Materials and Methods using a monoclonal antibody directed against the His₆ tag.

When protein synthesis was inhibited, the level of YebF in the periplasm gradually declined to a trace level while it progressively accumulated in the medium during the subsequent incubation period (FIG. 2B).

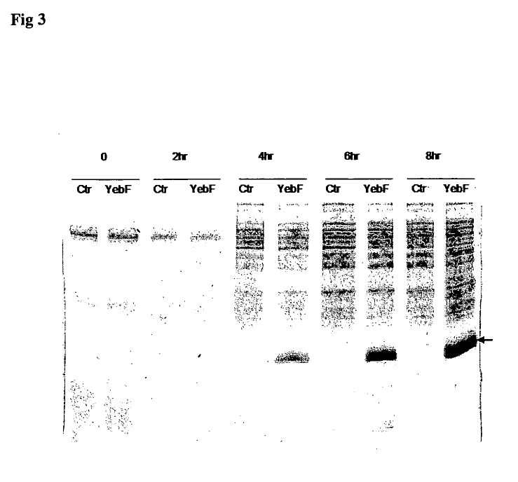

To demonstrate that YebF protein accumulates in the medium during growth, cells were induced with 0.1 mM IPTG, grown overnight, collected at the indicated time after induction and prepared as described in the Materials and Methods. As can be seen in FIG. 3, all the YebF was located in the culture medium.

When cells were induced and grown overnight, all the YebF was located in the medium (data not shown). Taken together, this data suggested that YebF is most likely an extracellular, soluble protein (see below).

Characteristics of the Secreted YebF

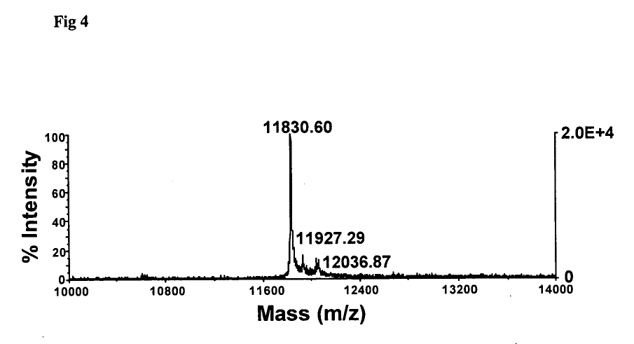

The secreted YebF-His₆ in both the periplasm and culture medium migrated on SDS-Tricine polyacrylamide gels at a molecular weight corresponding to the mature His-tagged protein (˜12 kD) (FIG. 3). YebF-His₆ was purified from the culture medium as described in the Materials and Methods and N-terminal amino acid sequence analysis revealed the sequence to be Ala-Asn-Asn-Glu-Thr-Ser-Lys-Ser-Val-Thr. This result indicates that YebF is cleaved immediately after the 21-amino acid sec-leader and not preceding the Cys at position −6 (FIG. 1). To examine whether there were any fatty acids bound to the mature YebF protein, the mass was determined to be 11,829.2+/−1.4 Daltons by Maldi-TOF linear mode mass spectrometry (FIG. 4). The calculated mass of mature His-tagged YebF with the 6-histidine tag and Leu-Glu linking amino acids, was 11,860.3 Daltons. This is 31.1 Daltons (0.26%) larger than the experimentally determined mass. If there were any covalently attached fatty chains, the mass determined by mass spectrometry would be expected to be larger than the calculated mass, suggesting therefore that YebF is not a lipoprotein.

Secretion of YebF into the Medium

The presence of YebF in the culture medium could be a result of cell lysis, leakage through the outer membrane, a natural cell secretion process or some combination. To test for lysis, immunoblotting was used to compare the subcellular localization of YebF and catabolite repressor protein (CRP), a well-known DNA binding protein residing in the cytoplasm. E. coli HB101/pYebFH₆/MS was grown in 50 ml of TB at 30° C. until OD₆₀₀ reached 0.6 and induced with 0.1 mM IPTG. Ten ml of cell culture was harvested at 2, 4 and 6 hours after induction. The cells were washed once in 10 ml of 100 mM MOPS and suspended in 10 ml of the same MOPS buffer. The medium was filtered through a 0.22 μm Millipore filter. Both cell suspension and medium were mixed with 2.5 ml of 25% trichloroacetic acid (TCA) to precipitate proteins. TCA trapped in the pellets was removed by two extractions using −20° C. acetone. Protein samples were loaded onto two parallel gels which were subsequently subjected to immunoblotting.

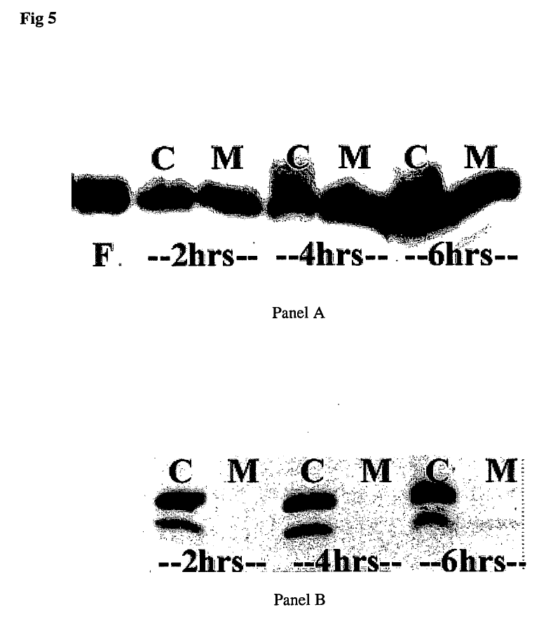

In cells, both CRP and YebF were readily detected. In the medium, YebF was detected at all experimental time points (FIG. 5A). CRP, however, was not observed in the medium at 2 and 4 hours after induction (FIG. 5B). A trace amount of CRP was detected at 6 hours, which indicates the beginning of inevitable cell lysis (FIG. 5). This indicates that the YebF found in the medium up to 4 hours post induction did not result from cell lysis.

In order to investigate the possible involvement of outer membrane leakage, two chimeric genes were constructed in the vector pMS119EH, under lac-promoter control, by fusing the leaderless, mature α-amylase gene from Bacillus subtilis⁹ to either the 3′ end of the yebF gene (pYebF-AmyH₆) or directly to the sec-leader sequence of yebF (pLS-AmyH₆). In addition, the leaderless mature α-amylase gene in the same vector (pAmyH₆) was used as a control. The mature α-amylase without any signal sequence should remain in the cytoplasm. Cells were grown in HB101 and induced as in FIG. 2. Cells were harvested 4 hours after induction with 0.1 mM IPTG, prior to any cell lysis and subjected to cold osmotic shock²¹ and α-amylase activity quantitated as described in Materials and Methods.

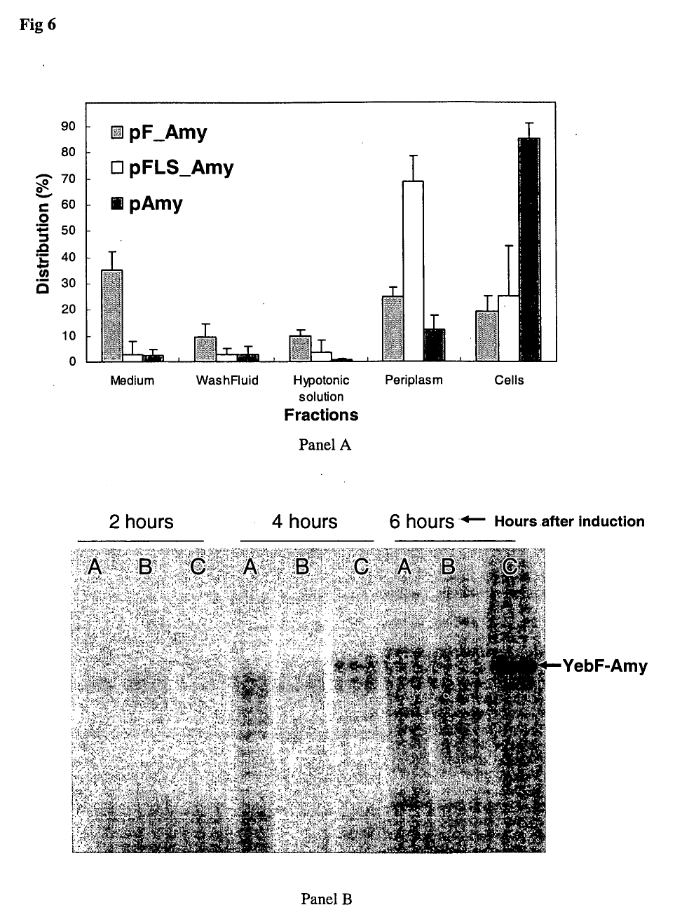

HB101 cells with pMS119EH produced an undetectable level of α-amylase either in the medium or in the cells (data not shown). Only α-amylase fused to full-length YebF could be found in the medium with 35% of the total activity in the medium. The leaderless α-amylase and α-amylase fused to the leader peptide of YebF were expressed but remained inside cells, and less than 5% of each on average were detected in the medium (FIG. 6A). These data were confirmed by the protein profile in the medium shown in FIG. 6B.

Using the cold osmotic shock method, the periplasm of cells was separated from the cytoplasm. As shown in FIG. 6A, over 68% of the α-amylase fused to the leader of YebF was recovered in the periplasm. 85% of leaderless α-amylase remained in the cytoplasm. 12% of the leaderless α-amylase found in the periplasm could be due to cell breakage during the cold osmotic shock. These data demonstrated that the YebF leader was able to direct the fusion protein from the cytoplasm to the periplasm using the sec-dependent translocation system but could not release the protein to the medium. Therefore these data together with the absence of α-amylase in the medium from cells harboring plasmid pLS-AmyH₆ indicate that the outer membrane was able to prevent mature α-amylase (the YebF leader peptide was removed by leader peptidase I) from leaking out of cells during normal growth.

The YebF-α-amylase fusion protein was able to cross the outer membrane from the periplasm (FIG. 6A). This demonstrates that YebF played an essential role in targeting the fusion protein for secretion across the outer membrane to the medium. In addition, FIG. 2 shows no apparent difference in the mobility of YebF protein in the periplasm or in the culture medium. This indicates that no additional cleavage occurred during secretion across outer membrane. This is also supported by the coincidence of the molecular weight calculated from the sequence (11,860 dalton) with that determined by mass spectrometry (11,829 dalton).

To further demonstrate that the presence of YebF in the medium is due to secretion from cells, rather than periplasmic leakage, the release of YebF-α-amylase was compared to the release of alkaline phosphatase, an endogenous well-defined periplasmic enzyme, in GZ39T/pYebF-AmyH₆ cells. Because alkaline phosphatase is induced by phosphate-starvation, it is very poorly expressed in wild-type E. coli cells grown in rich medium containing inorganic phosphate salts. To overcome this, a derivative of TG1, GZ39T, that expresses about 10 times more alkaline phosphatase than TG1 in rich medium, was isolated.

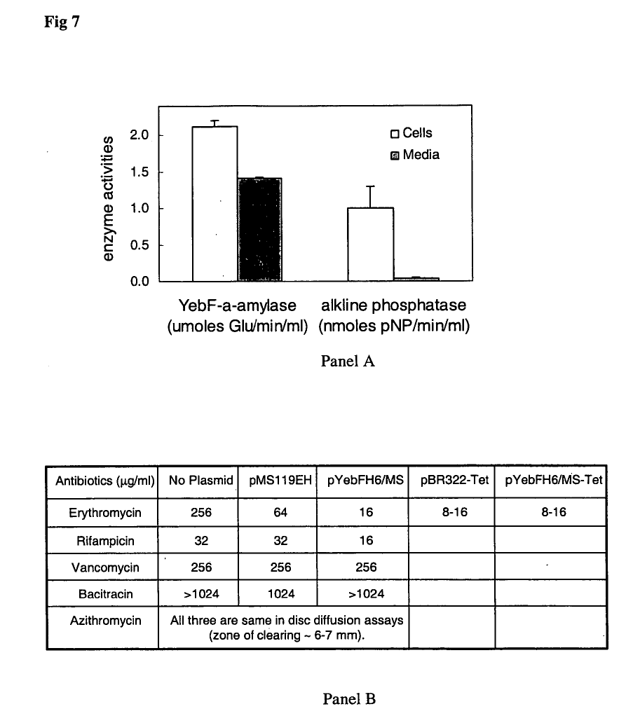

GZ39T/pYebF-AmyH₆ was grown in 25 ml of TB at 30° C. Cells were harvested at 7 hours after induction with 0.05 mM IPTG, washed with 100 mM MOPS (pH7.0) and suspended in 1.5 ml of 30 mM Tris-HCl (pH8.0). 200 μl of the cells were mixed with 300 μl of cell lysis buffer (see Methods and Materials). This cell lysate was used for both α-amylase and alkaline phosphatase activity assays. To measure α-amylase activity in the medium, 35 μl of the medium was directly used. To measure alkaline phosphatase activity, 8 ml of the medium was filtered three times through an Amicon ultracentrifugal filter (MWCO 30 KD) with 50 mM MOPS (pH7.0) to reduce phosphate and colored substances and finally concentrated to 0.6 ml. 200 μl of this preparation was used for the assay (see Methods and Materials). Alkaline phosphatase was assayed at 37° C. for 60 minutes in the dark. The results are shown in FIG. 7A, which shows that 42.6% of YebF-α-amylase was secreted to the medium compared to only 5.6% of alkaline phosphatase.

The possibility that over-expression of YebF causes destablization of the outer membrane was investigated by testing the permeability of 5 antibiotics: erythromycin, rifampicin, vancomycin, bacitracin and azithromycin. Little change in MICs (minimum inhibitory concentrations) was observed for cells expressing YebF or YebF-α-amylase in response to these agents, compared to cells either without plasmid or with pMS119EH (FIG. 7B).

Secretion Capability of Different E. coli Strains

3 ml of cells harboring pYebFH₆/MS or pYebFH₆/T7 were grown and induced as in FIG. 2A. Protein sample preparation and immunoblotting were conducted as described in the Materials and Methods. Monoclonal antibody against the histidine tag was used to monitor YebF-His₆.

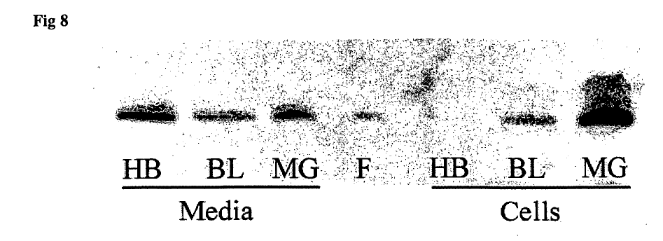

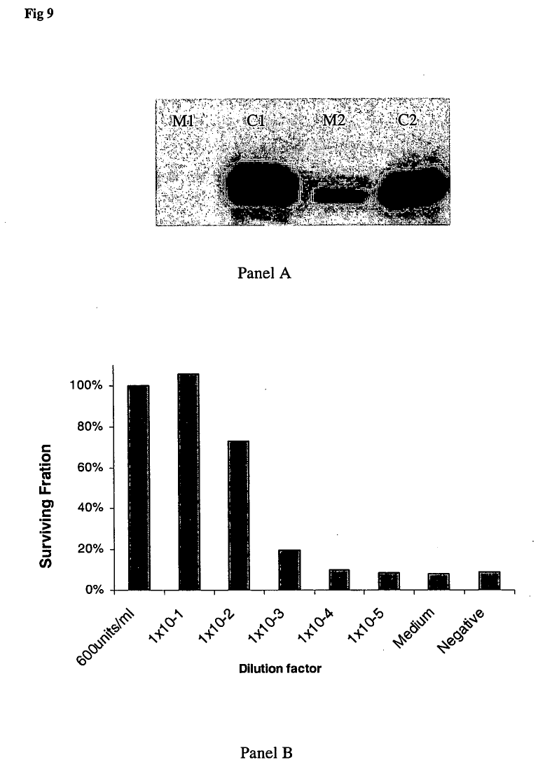

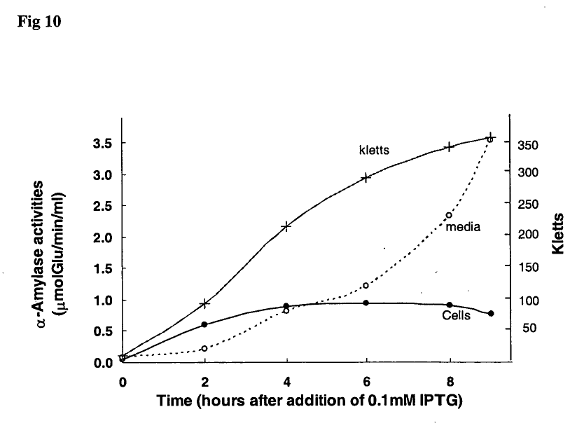

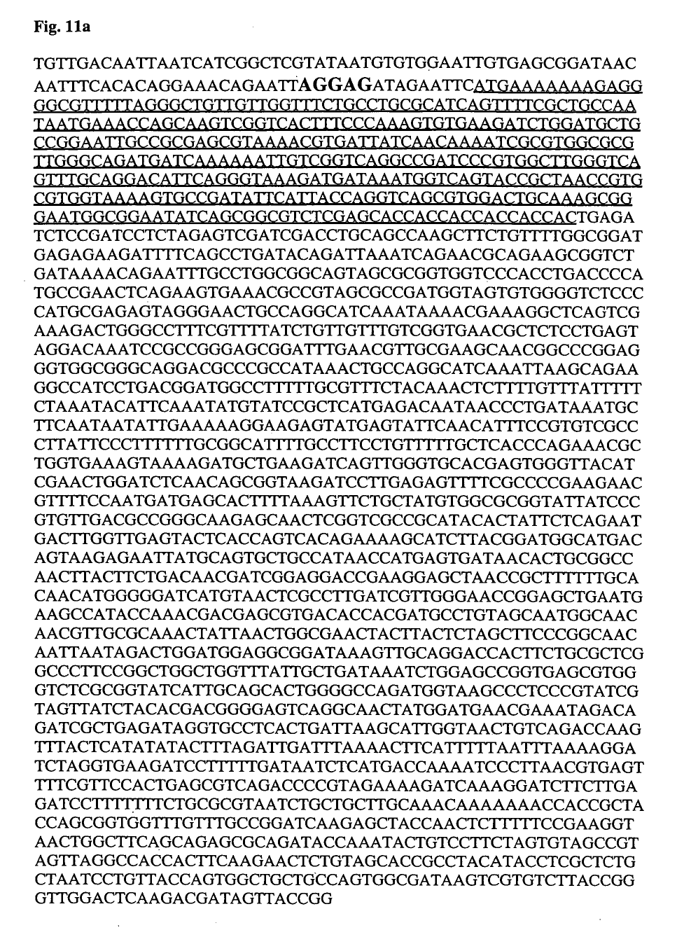

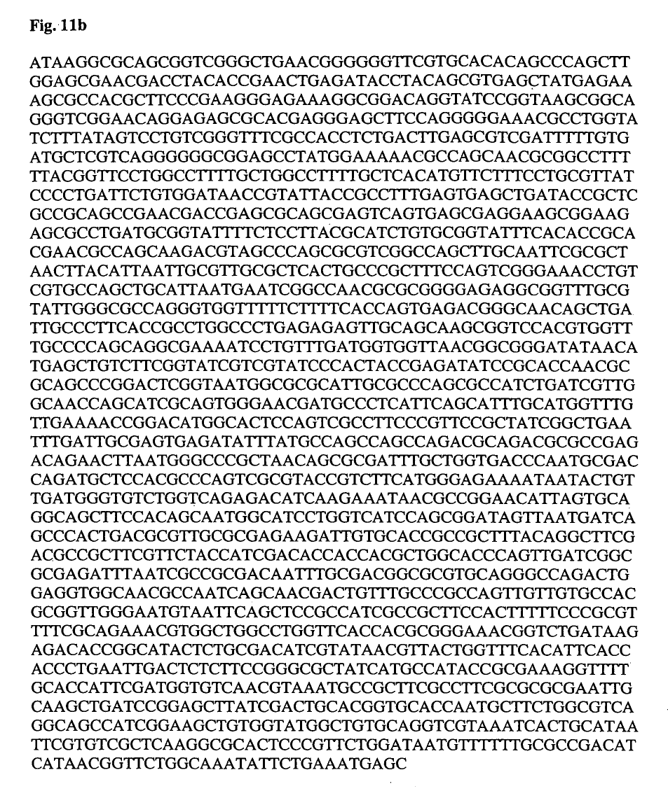

MG1655 (a K12 strain), BL21(DE3) (a B strain) and HB101 (a hybrid of K12 and B) all secrete YebF into the medium, although the efficiency varied (FIG. 8). This result may suggest that E. coli cells including common laboratory strains still possess the capability of secreting proteins including YebF to the surrounding milieu.

Recombinant Protein Production with YebF

Fusion protein gene constructs of leaderless α-amylase of B. subtilis X-23⁸ (48 kD), leaderless E. coli alkaline phosphatase (94 kD as a dimer) and the medically relevant protein human interleukin-2 (hIL-2) (15 kD) to the carboxy terminus of YebF were made as described in the Materials and Methods. These represent both hydrophilic (α-amylase and alkaline phosphatase) and hydrophobic (hIL-2) proteins. All three passenger proteins carried a His₆ tag at their C-terminus.

Cells comprising these fusion protein gene constructs were grown in 25 ml of TB at 30° C. 10 mM DTT was added to one culture at the same time as 0.05 mM IPTG was added to induce expression. Samples were taken 3 hours after induction.

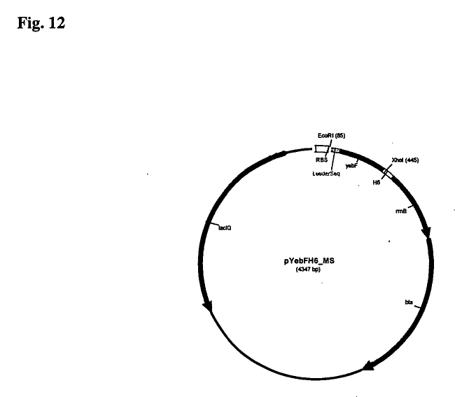

FIG. 9A shows the expression of mature YebF-hIL2 fusion protein in HB101/pYebF-hIL2H₆. The fusion protein was secreted to the medium at 3 hours after induction under regular growth conditions. With the presence of 10 mM DTT in the medium, however, cells did not secrete the fusion protein to the medium, although comparable expression inside cells was seen. Activity assays for the YebF-hIL-2 fusion protein based on the growth of CTLL-2 T-lymphocytes in vitro showed the secreted protein in the medium was active (FIG. 9B). The activity of the fusion protein was 43,800 units of hIL-2/ml of medium.

FIG. 10 shows that by 9 hours after induction 75% of mature YebF-α-amylase fusion protein was secreted from cells. It also appears that the fusion protein was stable in the medium and the maximal secretion of the YebF-α-amylase occurred after the culture reached stationary phase. From a 1-liter scale experiment, 31 mg of the purified YebF-α-amylase fusion protein with a specific activity of 380.7 μmol Glucose/min/mg protein was obtained, using nickel affinity chromatography. The specific activity of α-amylase in the initial culture medium varies with the time after induction from 150 mol Glucose/min/mg protein at 4 hours to 85 mole Glucose/min/mg protein at 9 hours after induction. It is 3.4 μmole Glucose/min/mg protein from B. subtilis culture⁴. The high specific activity here is also reflected in the simple protein profile of the medium shown in FIG. 6B.

Mature YebF-alkaline phosphatase fusion protein was also expressed from pYebF-phoAH₆/T7 plasmid in BL21(DE3) cells under the control of the T7 promoter. 23% was secreted to the medium at 7 hours after induction with IPTG.

### REFERENCES

The following references are cited in the application as numbers in brackets ([ ]) at the relevant portion of the application. Each of these references is incorporated herein by reference.
1. Lomba M R, et al. Identification of yebG as a DNA damage-inducible Escherichia coli gene. FEMS Microbiol. Lett. 156: 119-122 (1997).
2. Courcelle J, et al. Comparative gene expression profiles following UV exposure in wild-type and SOS-deficient Escherichia coli. Genetics. 158: 41-64 (2001).
3. van Dyk T K, et al. A genomic approach to gene fusion technology. Proc. Natl. Acad. Sci. USA. 98: 2555-2560 (2001).
4. Brokx S J, et al. Genome-Wide Analysis of Lipoprotein Expression in Escherichia coli MG1655. J. Bacteriol. 186: 3254-3258 (2004).
5. Shokri A, et al. Cell and process design for targeting of recombinant protein into the culture medium of Escherichia coli. Appl. Microbiol. Biotechnol. 60: 654-664 (2003).
6. Pugsley A P and Francetic O Protein secretion in Escherichia coli K-12: dead or alive? Cell Mol. Life Sci. 54: 347-52 (1998).
7. Errington J and Mountain A Is Bacillus an alternative expression system? In: Protein production by biotechnology. Edited by Harris T J R. Elsevier, pp. 1-15.(1990).
8. Ohdan K et al. Characteristics of two forms of alpha-amylases and structural implication. Appl. Environ. Microbiol. 65: 4652-4658 (1999).
9. Poquet I et al. Stable periplasmic secretion intermediate in the general secretory pathway of Escherichia coli. EMBO. J. 12: 271-278 (1993).
10. He S Y et al. Cloned Erwinia chrysanthemi out genes enable Escherichia coli to selectively secrete a diverse family of heterologous proteins to its milieu. Proc. Natl. Acad. Sci. USA. 88: 1079-1083 (1991).
11. Francetic O, et al. Expression of the endogenous type II secretion pathway in Escherichia coli leads to chitinase secretion. EMBO. J. 19: 6697-703 (2000).
12. Weiner J H, et al. A novel and ubiquitous system for membrane targeting and secretion of cofactor-containing proteins. Cell. 93:93-101 (1998).
13. Stathopoulos C, et al. Secretion of virulence determinants by the general secretory pathway in gram-negative pathogens: an evolving story. Microbes. Infect. 2: 1061-1072 (2000).
14. Francetic O and Pugsley A P The cryptic general secretory pathway (gsp) operon of Escherichia coli K-12 encodes functional proteins. J. Bacteriol. 178: 3544-3549 (1996).
15. Robb R J, Interleukin 2: the molecule and its function. Immunol. Today 5:203-209 (1984).
16. Masui Y, et al. Microheterogeneity of recombinant products: human interleukin 1α and 1β. Pp167-172. In: Curr. Commun. Mol. Biol.—Therapeutic peptides and proteins. Edited by Marshak D and Liu D. Cold Spring Harbor Laboratory Press, New York (1989).
17. Sambrook J and Russell D W Molecular cloning, a laboratory manual. 3^{rd} ed. Cold Spring Harbor Laboratory Press, Cold Spring Harbor, N.Y. (2001).
18. Strack B, et al. A common sequence Motif, -E-G-Y-A-T-A-, identified within the primase domains of plasmid-encoded I- and P-type DNA primases and the α protein of the Escherichia coli satellite phage P4. J. Biol. Chem. 267: 13062-13072 (1992).
19. Zhang G and Weiner J H CTAB-mediated purification of PCR products. Biotechniques 29: 982-986 (2000).
20. Barros E, et al., A novel cloning system for direct screening using a suicidal strategy. Gene. 179: 287-289 (1996).
21. Neu, H C and Heppel L A, The release of enzymes from Escherichia coli by osmotic shock and during the formation of spheroplasts. J. Biol. Chem. 240:3685-3692 (1965).
22. Schagger H and von Jagow G Tricine-sodium dodecyl sulfate-polyacrylamide gel electrophoresis for the separation of proteins in the range from 1 to 100 kDa. Anal. Biochem. 166: 368-379 (1987).
23. Lei H, et al. Induction of potent antitumor response by vaccination with tumor lysate-pulsed macrophages engineered to secrete macrophage colony-stimulating factor and interferon-g. Gene Ther. 7: 707-713 (2000).
24. Aurell L, et al. A New and Highly Specific Chromogenic Substrate for Factor Xa. Throm. Res. 11:595-6 09 (1977).
25. Ellinger S, et al. Cleavage of Procaryotically Expressed Human Immunodeficiency Virus Fusion Proteins by Factor Xa and Application in Western Blot (Immunoblot) Assays. J. Clin. Microbiol. 27:971-976 (1989).
26. Carrington J C and W G Dougherty A viral cleavage site cassette: identification of amino acid sequences required for tobacco etch virus polyprotein processing. Proc. Natl. Acad. Sci. USA 85: 3391-3395 (1988).
27. Walker P A et al. Efficient and rapid affinity purification of proteins using recombinant fusion proteases. Bio/Technology 12(6):601-5 (1994).
28. Cordingley M G. et al. Substrate requirements of human rhinovirus 3C protease for peptide cleavage in vitro. J. Biol. Chem. 265(16): 9062-5 (1990).
29. Chang J Y et al. Thrombin specificity. Selective cleavage of antibody light chains at the joints of variable with joining regions and joining with constant regions. Eur. J. Biochem. 151(2): 225-30 (1985).
30. Collins-Racie, L A et al. Production of recombinant bovine enterokinase catalytic subunit in Escherichia coli using the novel secretory fusion partner DsbA. Biotechnology, 13(9): 982-7 (1995).
31. Adelman J P et al. In vitro deletional mutagenesis for bacterial production of the 20,000-dalton form of human pituitary growth hormone. DNA 2(3):183-93 (1983).
32. Viera et al. Methods Enzymol. 153: 3 (1987)
33. Ausubel, Current Protocols in Molecular Biology, Eds. Ausubel et al. (John Wiley & Sons Inc NY) (1995-1999).
34. Zaccolo M et al., An approach to random mutagenesis of DNA using mixtures of triphosphate derivatives of nucleoside analogues. J. Mol. Biol. 255(4):589-603 (1996).
35. Stemmer W P DNA shuffling by random fragmentation and reassembly: in vitro recombination for molecular evolution Proc. Natl. Acad. Sci. USA 91(22):10747-51(1994).
36. Shafikhani S et al. Generation of large libraries of random mutants in Bacillus subtilis by PCR-based plasmid multimerization. Biotechniques 23(2):304-10 (1997).

We claim:

1. A method of producing a protein, polypeptide or peptide of interest that comprises:
(a) providing an expression vector that encodes a fusion protein comprising the protein, polypeptide or peptide of interest located carboxy-terminally to YebF, or a biologically active variant or portion thereof, and
(b) expressing the fusion protein in a suitable bacterial cell to thereby generate a secreted fusion protein.

2. The method of claim 1, further comprising the step of purifying the secreted fusion protein from the medium in which the bacterial cell is growing.

3. The method of claim 1, wherein the bacterial cell is Escherichia coli.

4. The method of claim 1, wherein the fusion protein further comprises at least one tag.

5. The method of claim 3, wherein the fusion protein further comprises at least one tag.

6. The method of claim 5, wherein the fusion protein further comprises at least one protein cleavage site.

7. A method of secreting a protein, polypeptide or peptide of interest from a bacterial cell into the growth medium comprising:
a) making an expression vector that comprises a recombinant DNA molecule that further comprises a nucleotide sequence that encodes the protein, polypeptide or peptide of interest joined in frame to a nucleotide sequence that encodes YebF, or a biologically active variant or portion thereof,
b) transfecting the expression vector into the bacterial cell, and
c) expressing the protein encoded by the recombinant DNA molecule in the bacterial cell under conditions that direct the secretion of the protein into the growth medium.

8. The method of claim 7, wherein the bacterial cell is Escherichia coli.

9. The method of claim 7 wherein the recombinant DNA molecule further comprises at least one nucleotide sequence that encodes a tag.

10. The method of claim 8 wherein the recombinant DNA molecule further comprises at least one nucleotide sequence that encodes a tag.

11. The method of claim 10 wherein the recombinant DNA molecule further comprises at least one nucleotide sequence that encodes a protein cleavage site.

12. The use of YebF, or a biologically active variant or portion thereof to direct the secretion of a protein, polypeptide or peptide of interest into bacterial growth medium.

13. The use of claim 12, wherein the YebF, or a biologically active variant or portion thereof is part of a fusion protein that further comprises the protein, polypeptide or peptide of interest.

14. The use of claim 13, wherein the YebF, or a biologically active variant or portion thereof is fused amino-terminally to the protein, polypeptide or peptide of interest.

15. An expression vector that encodes YebF, or a biologically active variant or portion thereof operatively linked to control nucleotide sequences that direct the transcription and the synthesis of the YebF or a biologically active variant or portion thereof.

16. The expression vector of claim 15 that encodes a fusion protein comprising YebF, or a biologically active variant or portion thereof and a protein, polypeptide or peptide of interest.

17. The expression vector of claim 16, wherein the nucleotide sequence that encodes the protein, polypeptide or peptide of interest is located downstream of the nucleotide sequence that encodes YebF, or a biologically active variant or portion thereof.

18. The expression vector of claim 17 for use in Escherichia coli.

19. The expression vector of claim 16 that encodes a fusion protein that further comprises at least one tag.

20. The expression vector of claim 19 that encodes a fusion protein further comprising at least one protein cleavage site.
